(19) United States
(12) Patent Application Publication
(10) Pub. No.: US 2026/0000668 A1
(43) Pub. Date: Jan. 1, 2026

(54) PREVENTION AND TREATMENT OF CORONAVIRUS INFECTION

(71) Applicant: ANAVEX LIFE SCENCES CORP., New York, NY (US)

(72) Inventor: Christopher U. Missling, New York, NY (US)

(21) Appl. No.: 18/552,305

(22) PCT Filed: Mar. 24, 2022
(86) PCT No.: PCT/US2022/021791

§ 371 Date: Sep. 25, 2023

Related U.S. Application Data
(60) Provisional application No. 63/165,542, filed on Mar. 24, 2021.

(51) Int. Cl. A61K 31/495; A61P 31/14

(52) U.S. Cl. CPC A61K 31/495; A61P 31/14

(57) ABSTRACT

Provided herein are compositions comprising one or more diphenyl compounds, such as ANAVEX 1066, or its metabolites, or isomers, or salts, for use in treating symptoms of, or preventing an infection by a coronavirus, particularly an infection caused by a SARS-COV-2 virus.

### CROSS REFERENCE TO RELATED APPLICATIONS

This application is a national phase application of International Application No. PCT/US2022/021971, filed Mar. 24, 2022, which claims the priority of U.S. provisional application No. 63/165,542 filed Mar. 24, 2021, the entire contents of which are herein incorporated by reference.

### FILED OF THE INVENTION

The present invention relates to the use of a diphenyl compound for inhibiting, treating and preventing coronavirus infection, and treating coronavirus disease.

### BACKGROUND OF THE INVENTION

Coronaviruses, members of the family Coronaviridae and subfamily Coronavirinae, are enveloped viruses containing single-strand, positive-sense RNA genome ranging from 26 to 32 kilobases in length. Coronaviruses have been identified in several vertebrate hosts including bird, bat, pig, rodent, camel, and human. Human can acquire coronavirus infection from other host of mammals, which may cause detrimental upper respiratory tract illness.

Members of the coronaviruses family include virus strains having different phylogenetic origin and causing different severity in mortality and morbidity. As such, treatment and prevention for coronavirus infection varies depending on the specific strains that cause the infection. Currently, we lack effective methods for prevention of coronavirus infection, and antiviral drug treatment once coronavirus infection has occurred. Accordingly, a need exists for the treatment and prevention of coronavirus infection, particularly of infection caused by SARS-COV-2, and COVID-19 disease.

### SUMMARY OF THE INVENTION

Provided herein is a method of inhibiting, preventing or treating an infection caused by a coronavirus, and a method of ameliorating or alleviating symptoms related with a coronavirus disease. The method may include administering a composition containing one or more diphenyl compounds to a subject in need thereof, wherein the diphenyl compounds may be a compound of Formula (I),

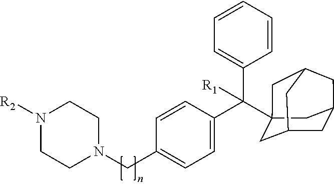

In Formula (I), n is an integer of 1, 2 or 3; R₁ and R₂ are each independently selected from the group consisting of hydrogen, C1-6 alkyl or C2-6 alkenyl, and C2-6 alkynyl. The diphenyl compound may also be, an isomer thereof, an enantiomer thereof, a racemic thereof, an intermediate thereof, a metabolite thereof, a pharmaceutically acceptable salt thereof, or a combination thereof. The isomer include, but not limited to, functional isomers, structural isomers, geometric isomers, optical isomers, enantiomeric isomers, and racemic isomers. The composition may be a pharmaceutical composition, and, in some instances, may further contain one or more pharmaceutically acceptable excipients. The composition may also be a nutraceutical composition, a health food composition, or a medical food composition. In one aspect, a diphenyl compound is ANAVEX 1066 with below structure,

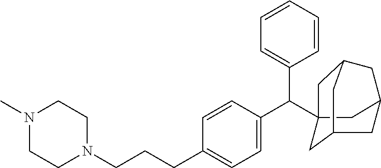 [SMILES: CN1CCN(CCCc2ccc(C(c3ccccc3)C34CC5CC(CC(C5)C3)C4)cc2)CC1]

or an isomer thereof, an enantiomer thereof, a racemic thereof, an intermediate thereof, a metabolite thereof, a pharmaceutically acceptable salt thereof, or a combination thereof, as described above. In yet one aspect, a diphenyl compound is ANAVEX 1066 dihydrochloride salt.

The diphenyl compound is administered in an amount sufficient to achieve a blood level for therapeutic or prophylactic effects. In particular, the blood level may be about 0.1-50 μg/ml. The amount of the diphenyl compound(s) administered to the subject may be from about 0.5 mg to about 1000 mg. In yet another aspect, the amount of the diphenyl compounds(s) administered to the subject may be from about 0.05 mg/kg to about 5 mg/kg.

The composition may be administered to the subject orally or via any parenteral route. The composition may be contained in a medical device, which may be an inhaler, a nebulizer, a nasal spray, or a vaporization aerosol device for administration to the subject. The composition may be administered at a frequency of three times per day to once per week, and for a period needed to exert and maintain the therapeutic or prophylactic effect, such as from two weeks to six months. In particular, the composition may be administered once or twice per day. Optionally, the subject may have undergone or may be undergoing treatment with one or more additional anti-viral agents.

The coronavirus may be a severe acute respiratory syndrome coronavirus 2 (SARS-COV-2), a severe acute respiratory syndrome coronavirus (SARS-COV), or a Middle East respiratory syndrome coronavirus (MERS-COV). The subject may be a mammal, such as cat, a dog, a cow, a duck, a pig, or a human being, who may test positive on coronavirus, and/or may be exhibiting the clinically recognized signs and symptoms of COVID-19, SARS or MERS.

Specifically, disclosed is a method for inhibiting or preventing a coronavirus infection, comprising administering to a subject in need thereof an effective amount of a diphenyl compound of Formula (I), an intermediate thereof, a metabolite thereof, an isomer thereof, an enantiomer thereof, an ester thereof, a pharmaceutically acceptable salt thereof, and a combination thereof. In one aspect, the effective amount of the diphenyl compound is sufficient to inhibit replication or proliferation of a coronavirus. In another aspect, the effective amount is sufficient to inhibit both replication and proliferation of a coronavirus. In yet another aspect, the subject has or is suspected of having a coronavirus infection caused by a severe acute respiratory syndrome coronavirus 2 (SARS-COV-2), a severe acute respiratory syndrome coronavirus (SARS-COV), a Middle East respiratory syndrome coronavirus (MERS-CoV), or any combination thereof. In one aspect, the diphenyl compound is ANAVEX 1066, or a metabolite, an enantiomer, an isomer, or a pharmaceutically acceptable salt thereof.

Also disclosed is a method for treating a coronavirus disease in a subject, comprising administering to the subject in need thereof an effective amount of a diphenyl compound of Formula (I) or an intermediate, a metabolite, an enantiomer, an isomer, an ester, or a pharmaceutically acceptable salt of any of the foregoing compounds, or any combination thereof. In one aspect, the subject being treated has or is suspected of having a coronavirus disease selected from severe acute respiratory syndrome (SARS), coronavirus disease 2019 (COVID-19), Middle East respiratory syndrome (MERS), or a combination thereof. In one aspect, the diphenyl compound is ANAVEX 1066, its metabolites, its isomer, or its pharmaceutically acceptable salt thereof.

Also disclosed is a method for reducing the risk of contracting a coronavirus infection or developing a coronavirus disease in a subject, comprising administering to the subject in need thereof an effective amount of a diphenyl compound of Formula (I). In one aspect, the diphenyl compound is selected from the group consisting of an intermediate thereof, a metabolite thereof, an enantiomer, an isomer thereof, an ester thereof, a pharmaceutically acceptable salt thereof, and a combination thereof. In yet another aspect, the effective amount is sufficient to prevent or prohibit the replication or proliferation of a coronavirus. In yet another aspect, the coronavirus is a severe acute respiratory syndrome coronavirus 2 (SARS-COV-2), a severe acute respiratory syndrome coronavirus (SARS-COV), a Middle East respiratory syndrome coronavirus (MERS-COV), or a combination thereof.

Also disclosed is a method for alleviating or ameliorating symptoms related to a coronavirus disease in a subject, comprising administering to the subject in need thereof an effective amount of a diphenyl compound of Formula (I), such as ANAVEX 1066, an intermediate, a metabolite, an enantiomer, an isomer, an ester, or a pharmaceutically acceptable salt of any of the foregoing compounds, or a combination thereof. In one aspect, the subject has shown or is indicative of symptoms related to a coronavirus disease. The symptoms may include, but not limited to, fever or chills, cough, shortness of breath or difficulty breathing, fatigue, muscle or body aches, headache, loss of taste or smell, sore throat, congestion or runny nose, nausea or vomiting, diarrhea, trouble breathing, persistent pain or pressure in the chest, new confusion, inability to wake or stay awake, bluish lips or face. The coronavirus disease is selected from severe acute respiratory syndrome (SARS), coronavirus disease 2019 (COVID-19), Middle East respiratory syndrome (MERS), or a combination thereof. In one aspect, the diphenyl compound is ANAVEX 1066, its metabolites, its enantiomer, its isomer, or its pharmaceutically acceptable salt thereof.

In one aspect, for all the methods disclosed, the diphenyl compound is administered orally or parenterally. In another aspect, the diphenyl compound is administered in a composition in the form of a pharmaceutical composition, a health food composition, or a medical food composition. In yet another aspect, a pharmaceutical composition as disclosed includes compositions which further comprise one or more pharmaceutically acceptable carriers and/or excipients.

In one aspect, the subject mentioned above is a mammal. In yet another aspect, the mammal is a human being, a cat, a dog, a cow, a pig, a duck or a chicken. In yet another aspect, the human being is less than 20 years old or over 50 years old.

In one aspect, the amount of the diphenyl compound(s) administered to the subject is from about 0.5 to about 1000 mg. In one aspect, the amount of the diphenyl compound(s) administered to the subject is from about 0.05 mg/kg to about 5 mg/kg. In another aspect, the amount is about 1 mg per day to about 250 mg per day, as well as from about 5 mg per day to about 50 mg per day and more particularly from about 10/mg per day to about 30 mg per day. Yet another aspect is for dosages of from about 0.5 mg to about 1000 mg, and more particularly from about 0.5 mg to about 5 mg; from about 5 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 400 mg, from about 400 mg to about 500 mg, and from 500 mg to about 600 mg, and from 600 mg to about 750 mg, from about 750 mg to about 1000 mg. For the diphenyl compound with one or more chiral center(s), dosing with the (+) form substantially absent the (−) form is noted. In specific embodiments dosing may be with the (−) form substantially absent the (+) form. “Substantially absent” shall be understood to mean no more than about 5% w/w and more preferably no more than 1% w/w. In another aspect, the diphenyl compound is administered to the subject at a frequency ranging from as often as three times per day, to once per week, specific references made to two times per day, one time per day, five times per week, four times per week, three times per week, two times per week, and one time per week. In another aspect, the diphenyl compound is administered to the subject for a total period of about two weeks to about six months. In yet another aspect, the subject has previously undergone, or is also undergoing treatment with one or more anti-viral agents in addition to treatment with the diphenyl compound.

The details of one or more aspects of the invention are set forth in the description below. Other features or advantages of the present invention will be apparent from the following drawings and detailed description of several aspects, and also from the appended claims.

### BRIEF DESCRIPTION OF THE DRAWINGS

In order to describe the manner in which the advantages and features of the disclosure can be obtained, reference is made to aspects thereof which are illustrated in the appended drawings. Understanding that these drawings depict only exemplary aspects of the disclosure and are not therefore to be considered to be limiting of its scope, the principles herein are described and explained with additional specificity and detail through the use of the accompanying drawings.

FIG. 1 is a schematic of the synthesis of AV1066.

FIG. 2 is a plot of viral titer reduction measured by “Mean Log10 Reduction” against the concentrations of ANAVEX1066.

FIG. 3 is a plot of “Percent Protection” against the log concentrations of ANAVEX1066 and remdesivir.

### DETAILED DESCRIPTION

The present disclosure is based, at least in part, on the surprising discovery that the diphenyl compound ANAVEX 1066 is effective in inhibiting, prohibiting or preventing coronavirus (e.g., SARS-COV2) proliferation and/or replication in vitro, thereby indicating therapeutic benefit in treating an infection caused by a coronavirus. Accordingly, provided herein are methods for inhibiting, preventing, treating, alleviating, ameliorating and reducing the risk of a coronavirus infection using a composition comprising one or more the diphenyl compounds. The methods include administering to a subject in need of the treatment (e.g., a human patient having an infection caused by a coronavirus) an effective amount of a diphenyl compound or a composition comprising such.

### Diphenyl Compound

As used herein, the term “diphenyl compound” refers to a compound having a structure moiety shown in Formula (I), and a derivative, an intermediate, a metabolite, an analog or a pharmaceutically acceptable salt, an ester, or an isomer thereof,

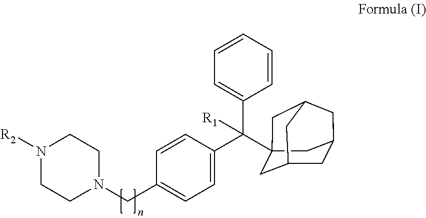

wherein the two phenyl groups are connected to the same carbon, wherein the carbon can be a chain or a ring carbon, and wherein the carbon has a substituent R₁. Suitable R₁ may be hydrogen, hydroxyl, C1-6 alkyl, C2-6 alkenyl or C2-6 alkynyl. Further, n is an integer selected from 1, 2, or 3, and the substituent R₂ includes, but are not limited to, alkyls, alkenyls, alkynyls, amines, amides, aromatic rings, non-aromatic rings, halogens, —CN, —NO₂, —N₃, OR, —NH₂, or —SR, R being hydrogen, halogen, —CN, —NO₂, —N₃, acyl, alkyl, alkenyl, and alkynyl. Some unlimited examples of the diphenyl compound are shown in Table 1.

TABLE 1
Examples of Diphenyl Compound

| CAS | | |
|---|---|---|
| Reg. No. | Chemical Name | Structure |
| 1410790- 33-8 | Piperazine, 1-(phenylmethyl)-4-[3-[4- (phenyltricyclo[3.3.1.1^{3,7}]dec-1- ylmethyl)phenyl]propyl]-, compd. with 2,4,6-trinitrophenol (1:2) | 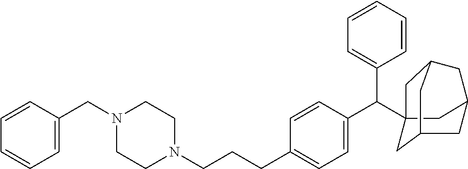 [SMILES: c1ccc(CN2CCN(CCCc3ccc(C(c4ccccc4)C45CC6CC(CC(C6)C4)C5)cc3)CC2)cc1] |
| | | 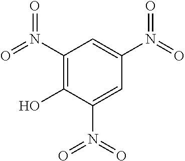 [SMILES: O=[N+]([O-])c1cc([N+](=O)[O-])c(O)c([N+](=O)[O-])c1] |
| 1410790- 31-6 | Piperazine, 1-(phenylmethyl)-4-[3-[4- (phenyltricyclo[3.3.1.1^{3,7}]dec-1- ylmethyl)phenyl]propyl]-, hydrochloride (1:2) | 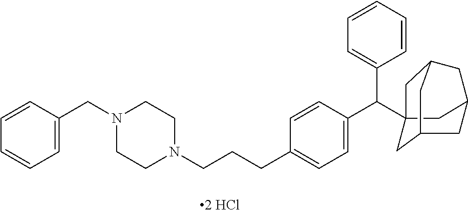 [SMILES: c1ccc(CN2CCN(CCCc3ccc(C(c4ccccc4)C45CC6CC(CC(C6)C4)C5)cc3)CC2)cc1] |
| 1410790- 29-2 | Piperazine, 1-ethyl-4-[3-[4- (phenyltricyclo[3.3.1.1^{3,7}]dec-1- ylmethyl)phenyl]propyl]-, hydrochloride (1:2) | 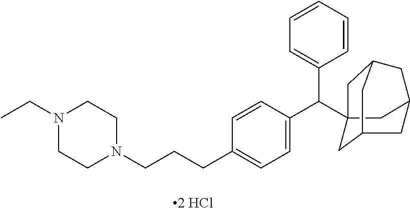 [SMILES: CCN1CCN(CCCc2ccc(C(c3ccccc3)C34CC5CC(CC(C5)C3)C4)cc2)CC1] |
| 1410790- 23-6 | Piperazine, 1-[3-[4- (phenyltricyclo[3.3.1.1^{3,7}]dec-1- ylmethyl)phenyl]propyl]- | 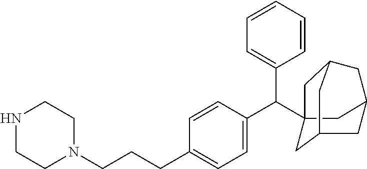 [SMILES: c1ccc(C(c2ccc(CCCN3CCNCC3)cc2)C23CC4CC(CC(C4)C2)C3)cc1] |
| 1410790- 21-4 | Piperazine, 1-(phenylmethyl)-4-[3-[4- (phenyltricyclo[3.3.1.1^{3,7}]dec-1- ylmethyl)phenyl]propyl]- | 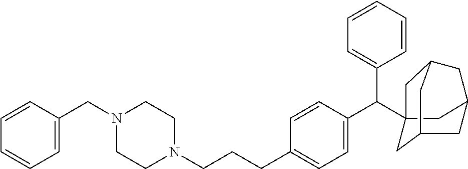 [SMILES: c1ccc(CN2CCN(CCCc3ccc(C(c4ccccc4)C45CC6CC(CC(C6)C4)C5)cc3)CC2)cc1] |
| 1410790- 20-3 | Piperazine, 1-ethyl-4-[3-[4- (phenyltricyclo[3.3.1.1^{3,7}]dec-1- ylmethyl)phenyl]propyl]- | 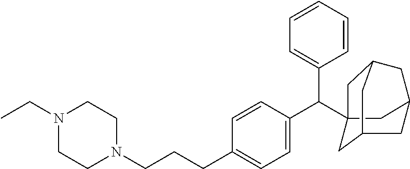 [SMILES: CCN1CCN(CCCc2ccc(C(c3ccccc3)C34CC5CC(CC(C5)C3)C4)cc2)CC1] |
| 1410790- 15-6 | Piperazine, 1-ethyl-4-[2-[4- (phenyltricyclo[3.3.1.1^{3,7}]dec-1- ylmethyl)phenyl]ethyl]-, hydrochloride (1:2) | 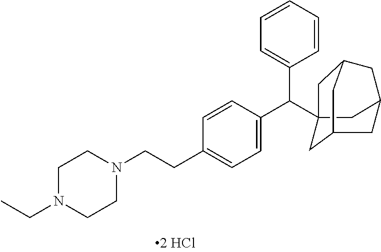 [SMILES: CCN1CCN(CCc2ccc(C(c3ccccc3)C34CC5CC(CC(C5)C3)C4)cc2)CC1] |
| 1410790- 13-4 | Piperazine, 1-methyl-4-[2-[4- (phenyltricyclo[3.3.1.1^{3,7}]dec-1- ylmethyl)phenyl]ethyl]-, hydrochloride (1:2) | 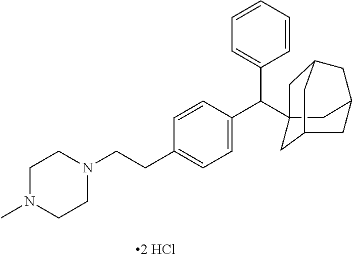 [SMILES: CN1CCN(CCc2ccc(C(c3ccccc3)C34CC5CC(CC(C5)C3)C4)cc2)CC1] |
| 1410790- 08-7 | Piperazine, 1-ethyl-4-[2-[4- (phenyltricyclo[3.3.1.1^{3,7}]dec-1- ylmethyl)phenyl]ethyl]- | 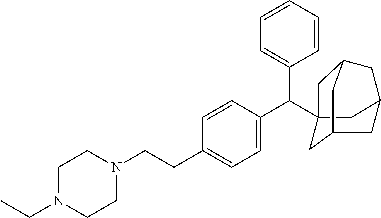 [SMILES: CCN1CCN(CCc2ccc(C(c3ccccc3)C34CC5CC(CC(C5)C3)C4)cc2)CC1] |
| 1410790- 06-5 | Piperazine, 1-methyl-4-[2-[4- (phenyltricyclo[3.3.1.1^{3,7}]dec-1- ylmethyl)phenyl]ethyl]- | 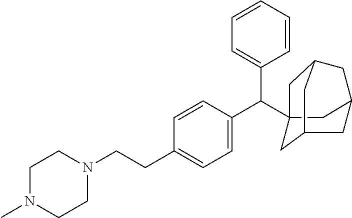 [SMILES: CN1CCN(CCc2ccc(C(c3ccccc3)C34CC5CC(CC(C5)C3)C4)cc2)CC1] |
| 1410790- 01-0 | Piperazine, 1-[[4- (phenyltricyclo[3.3.1.1^{3,7}]dec-1- ylmethyl)phenyl]methyl]-, hydrochloride (1:2) | 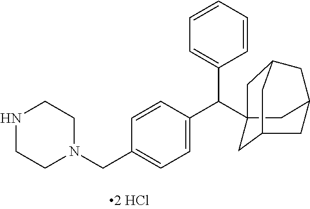 [SMILES: c1ccc(C(c2ccc(CN3CCNCC3)cc2)C23CC4CC(CC(C4)C2)C3)cc1] |
| 1410789- 99-9 | Piperazine, 1-ethyl-4-[[4- (phenyltricyclo[3.3.1.1^{3,7}]dec-1- ylmethyl)phenyl]methyl]-, hydrochloride (1:2) | 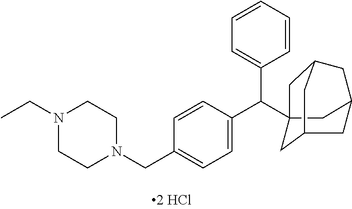 [SMILES: CCN1CCN(Cc2ccc(C(c3ccccc3)C34CC5CC(CC(C5)C3)C4)cc2)CC1] |
| 1410789- 97-7 | Piperazine, 1-methyl-4-[[4- (phenyltricyclo[3.3.1.1^{3,7}]dec-1- ylmethyl)phenyl]methyl]-, hydrochloride (1:2) | 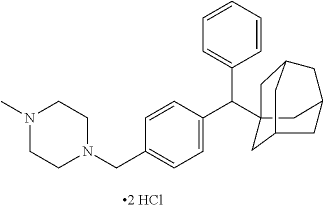 [SMILES: CN1CCN(Cc2ccc(C(c3ccccc3)C34CC5CC(CC(C5)C3)C4)cc2)CC1] |
| 1410789- 93-3 | Piperazine, 1-[[4- (phenyltricyclo[3.3.1.1^{3,7}]dec-1- ylmethyl)phenyl]methyl]- | 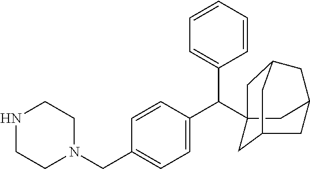 [SMILES: c1ccc(C(c2ccc(CN3CCNCC3)cc2)C23CC4CC(CC(C4)C2)C3)cc1] |
| 1410789- 90-0 | Piperazine, 1-ethyl-4-[[4- (phenyltricyclo[3.3.1.1^{3,7}]dec-1- ylmethyl)phenyl]methyl]- | 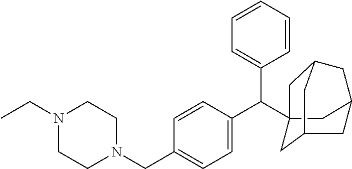 [SMILES: CCN1CCN(Cc2ccc(C(c3ccccc3)C34CC5CC(CC(C5)C3)C4)cc2)CC1] |
| 1410789- 88-6 | Piperazine, 1-methyl-4-[[4- (phenyltricyclo[3.3.1.1^{3,7}]dec-1- ylmethyl)phenyl]methyl]- | 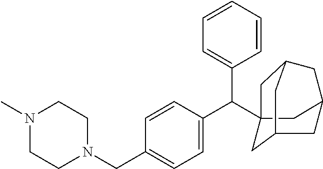 [SMILES: CN1CCN(Cc2ccc(C(c3ccccc3)C34CC5CC(CC(C5)C3)C4)cc2)CC1] |
| 1410789- 68-2 | Ethanone, 1-(4-methyl-1- piperazinyl)-2-[4- (phenyltricyclo[3.3.1.1^{3,7}]dec-1- ylmethyl)phenyl]- | 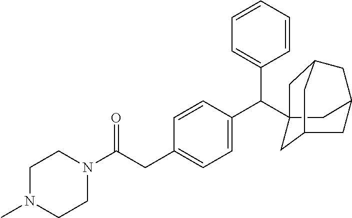 [SMILES: CN1CCN(C(=O)Cc2ccc(C(c3ccccc3)C34CC5CC(CC(C5)C3)C4)cc2)CC1] |
| 1410789- 60-4 | Tricyclo[3.3.1.1^{3,7}]decane-1- methanol, α-[4-[2-(4-ethyl-1- piperazinyl)ethyl]phenyl]-α-phenyl- | 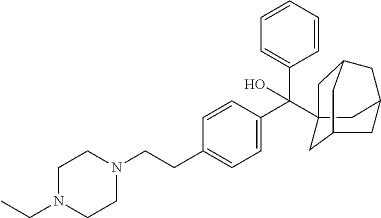 [SMILES: CCN1CCN(CCc2ccc(C(O)(c3ccccc3)C34CC5CC(CC(C5)C3)C4)cc2)CC1] |
| 1410789- 59-1 | Tricyclo[3.3.1.1^{3,7}]decane-1- methanol, α-[4-[2-(4-methyl-1- piperazinyl)ethyl]phenyl]-α-phenyl- | 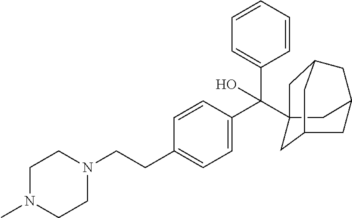 [SMILES: CN1CCN(CCc2ccc(C(O)(c3ccccc3)C34CC5CC(CC(C5)C3)C4)cc2)CC1] |
| 1410789- 51-3 | Tricyclo[3.3.1.1^{3,7}]decane-1- methanol, α-phenyl-α-[4-(1- piperazinylmethyl)phenyl]- | 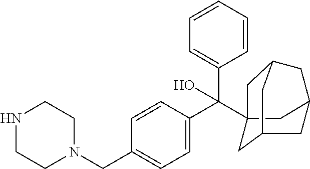 [SMILES: OC(c1ccccc1)(c1ccc(CN2CCNCC2)cc1)C12CC3CC(CC(C3)C1)C2] |
| 1410789- 50-2 | Tricyclo[3.3.1.1^{3,7}]decane-1- methanol, α-[4-[(4-ethyl-1- piperazinyl)methyl]phenyl]-α-phenyl- | 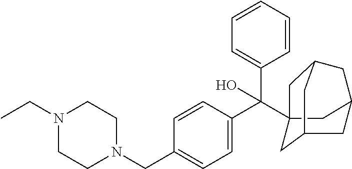 [SMILES: CCN1CCN(Cc2ccc(C(O)(c3ccccc3)C34CC5CC(CC(C5)C3)C4)cc2)CC1] |
| 1410789- 49-9 | Tricyclo[3.3.1.1^{3,7}]decane-1- methanol, α-[4-[(4-methyl-1- piperazinyl)methyl]phenyl]-α-phenyl- | 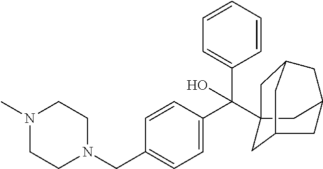 [SMILES: CN1CCN(Cc2ccc(C(O)(c3ccccc3)C34CC5CC(CC(C5)C3)C4)cc2)CC1] |
| 1041003- 37-5 | 1-Methyl-4-[3-[4- (phenyltricyclo[3.3.1.1^{3,7}]dec-1- ylmethyl)phenyl]propyl]piperazine | 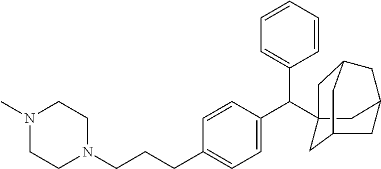 [SMILES: CN1CCN(CCCc2ccc(C(c3ccccc3)C34CC5CC(CC(C5)C3)C4)cc2)CC1] ANAVEX 1066 |
| 1040716- 94-6 | Piperazine, 1-methyl-4-[3-[4- (phenyltricyclo[3.3.1.1^{3,7}]dec-1- ylmethyl)phenyl]propyl]-, hydrochloride (1:2) | 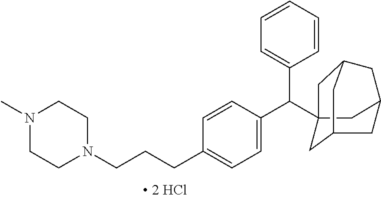 [SMILES: CN1CCN(CCCc2ccc(C(c3ccccc3)C34CC5CC(CC(C5)C3)C4)cc2)CC1] ANAVEX 1066 dihydrochloride salt |

In one aspect, the diphenyl compound may be ANAVEX 1066, or a derivative, an intermediate, a metabolite, an ester, an enantiomer, an isomer or a salt thereof. Isomers include, but are not limited to functional isomers, structural isomers, geometric isomers, optical isomers, enantiomeric isomers, and racemates.

Provided herein are compositions comprising one or more of the diphenyl compounds disclosed herein, for use in treating coronavirus infection. The composition may be a pharmaceutical composition, a nutraceutical composition, a health food composition, or a medical food composition. Any of the diphenyl compounds may be prepared by routine chemical synthesis routes such as those disclosed in Chemische Berichte (1994), 127(3), 557-63 and Journal of Medicinal Chemistry (2012), 55 (22), 10241-10261, the entire disclosure of each of which is incorporated herein by reference.

It will be understood that any of the compositions may comprise one or more diphenyl compounds, or an intermediate, a metabolite, an ester, an enantiomer, an isomer or a salt thereof as described herein. For example, such a composition may comprise the one or more diphenyl compounds as the only anti-coronavirus agent(s). Alternatively, such a composition may comprise two or more diphenyl compounds, and/or include a second antiviral or other therapeutic agent.

### Pharmaceutical Compositions

Any composition that can carry the effective amount of the diphenyl compounds and can effectively be administered to the subject in need thereof is within the scope of the current invention. In some aspects, the compositions described herein can be a pharmaceutical composition, which may further comprise a pharmaceutically acceptable carrier and/or excipient. Pharmaceutically acceptable carriers include diluents, fillers, salts, buffers, stabilizers, solubilizers and other material which are well-known in the art. Such compositions may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from a suitable inorganic base, (e.g., sodium hydroxide, barium hydroxide, iron (ii) hydroxide, iron (III) hydroxide, magnesium hydroxide, calcium hydroxide, aluminum hydroxide, ammonium hydroxide, potassium hydroxide, caesium hydroxide, or lithium hydroxide) or a suitable organic base (e.g., pyridine, methyl amine, imidazole, benzimidazole, histidine, phosphazene bases, or a hydroxide of an organic cation such as quaternary ammonium hydroxide and phosphonium hydroxide). Also, pharmaceutically-acceptable salts can be prepared as alkaline metal, alkaline earth or other conjugating salts, such as lithium, sodium, potassium, calcium, ammonium, phosphate, hydrochloride, dihydrochloride or mesylate salts.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbate, poloxamers, or polyethylene glycol (PEG).

In some examples, the pharmaceutical composition described herein includes pulmonary compatible excipients. Suitable such excipients include, but not limited to, richloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane, purified water, ethanol, propylene glycol, glycerin, PEG (e.g. PEG400, PEG 600, PEG 800 and PEG 1000), sorbitan trioleate, soya lecithin, lecithin, oleic acid, Polysorbate 80, magnesium stearate and sodium lauryl sulfate, methylparaben, propylparaben, chlorobutanol, benzalkonium chloride, cetylpyridinium chloride, thymol, ascorbic acid, sodium bisulfite, sodium metabisulfite, EDTA, sodium hydroxide, tromethamine, ammonia, HCl, H₂SO₄, HNO₃, citric acid, CaCl₂), CaCO₃, sodium citrate, sodium chloride, disodium EDTA, saccharin, menthol, ascorbic acid, glycine, lysine, gelatin, povidone K25, silicon dioxide, titanium dioxide, zinc oxide, lactose, lactose monohydrate, lactose anhydrate, mannitol, and dextrose. The composition may be contained in a medical device, which may be an inhaler, a nebulizer, a nasal spray, or a vaporization aerosol device for administration to the subject.

In other examples, the pharmaceutical composition described herein can be formulated in controlled time release forms, such as compositions for immediate release or sustained-release. Suitable examples of sustained-release preparations include semipermeable matrices of solid hydrophobic polymers which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle or a sealed container to be manually accessed.

The pharmaceutical compositions described herein can be in unit dosage forms such as solids, solutions or suspensions, or suppositories, for administration by inhalation or insufflation, intrathecal, intrapulmonary or intracerebral routes, oral, parenteral or rectal administration.

For preparing solid compositions, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as powder collections, tablets, pills and capsules. This solid pre-formulation composition is then subdivided into unit dosage forms of the type described above containing a suitable amount of the active ingredient in the composition.

Suitable surface-active agents include, in particular, non-ionic agents, such as polyoxyethylene sorbitans, and polysorbates. Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID®, LIPOSYN®, INFONUTROL®, LIPOFUNDIN® and LIPIPHYSAN®. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some aspects, the compositions are administered by the oral or nasal respiratory route for local or systemic effect. In some aspects, the compositions are composed of particle sized between 10 nm to 100 mm.

Compositions in preferably sterile pharmaceutically acceptable solvents may be nebulized by use of gases. Nebulized solutions may be breathed directly from the nebulizing device or the nebulizing device may be attached to a face mask, tent, endotracheal tube and/or intermittent positive pressure breathing machine (ventilator). Solution, suspension or powder compositions may be administered, preferably orally or nasally, from devices which deliver the formulation in an appropriate manner.

The pharmaceutical compositions provided herein can be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain aspects, the chronic administration is intended to provide a constant level of the compound in the blood, e.g., within the therapeutic window over the extended period of time.

The compositions for oral administration can take the form of tablets, capsules, caplets, bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form. For example, the diphenyl compounds may be administered to the subject in a dose of 0.5 mg to 1,000 mg. In another example, the dose of the diphenyl compounds may range from about 0.05 mg per kg to about 5 mg per kg of the subject's body weight (mg/kg), for example, about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 2, 3, 4 or 5 mg/kg. The dose may also be at least one of the foregoing amounts or a range thereof. In one example, the dose may be about 1 mg/kg.

Liquid forms suitable for oral administration may include a suitable aqueous or non-aqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also, In some aspects, be administered by a transdermal device. Accordingly, transdermal administration can be accomplished using a patch either of the reservoir or porous membrane type, or of a solid matrix variety. The noted components for orally administrable, injectable or topically administrable compositions are merely representative. Other materials as well as processing techniques and the like are set forth in Remington: The Science and Practice of Pharmacy, 22nd Edition (2012), Pharmaceutical Press, which is incorporated herein by reference. The compounds of this invention can also be administered in sustained release forms or from sustained release drug delivery systems.

In some aspects, any of the pharmaceutical compositions herein may further comprise a second therapeutic agent based on the intended therapeutic uses of the composition. Specifically, the second therapeutic agent can be another agent that is active in inhibiting, preventing, or treating a viral infection, or an agent capable of alleviating or ameliorating the symptoms of a viral disease.

To achieve better compliance in terms of administering the composition on time and for the full length of prescribed period, special attention is called out to consider the subject's factors when selecting a specific composition for the subject, such as subject's physical and mental conditions (whether they can self-administer), con-current disorders (for example, diabetic patients should avoid compositions containing extra-sugar), specific allergies, and personal preferences (liquid over solid, oral over injection).

### Nutraceuticals and Food Compositions

In some aspects, the compositions described herein can be a food composition such as a health food and/or nutraceutical composition. Such food compositions may be in any liquid or solid/semi-solid form such as those used for nourishing humans and animals, for treatment of virus infection, or in particular, coronavirus infection. Food compositions may be beverages or solid/semi-solid food, including but not limited to tea-based beverages, juices, soft drinks, coffees, milks, jellies, cookies, cereals, chocolates, snack bars, herbal extracts, dairy products (e.g., ice cream, and yogurt)), or may take the form of a food/dietary supplement as a tablet, capsule, powder or gummy.

Food compositions described herein may comprise one or more edible carriers, which confer one or more of the benefits to the composition in the product as described herein. Examples of edible carriers include starch, cyclodextrin, maltodextrin, methylcellulose, carbonmethoxy cellulose, xanthan gum, and aqueous solutions thereof. Other examples include solvents, dispersion media, coatings, surfactants, antioxidants, preservatives (e.g., antibacterial agents, antifungal agents), isotonic agents, absorption delaying agents, stabilizers, gels, binders, excipients, disintegration agents, lubricants, sweetening agents, flavoring agents, dyes, such like materials and combinations thereof, as would be known to one of ordinary skill in the art. In some examples, the healthy food products described herein may further include neuroprotective foods, such as fish oil, flax seed oil, and/or benzoate.

A nutraceutical composition refers to compositions containing components from food sources and conferring extra health benefits in addition to the basic nutritional value found in foods. A nutraceutical composition as described herein comprises a composition described herein and additional ingredients and supplements that promote good health and/or enhance stability and bioactivity.

Activity of nutraceutical compositions may be fast or/and short-term or may help achieve long-term health objectives as those described herein, e.g., improving health conditions, in, e.g., human subjects who have or are at risk for virus infection. The nutraceutical compositions may be contained in an edible material, for example, as a dietary supplement or a pharmaceutical formulation. As a dietary supplement, additional nutrients, such as vitamins, minerals or amino acids may be included. The composition can also be a drink or a food product, e.g., tea, soft drink, juice, milk, coffee, cookie, cereal, chocolate, and snack bar. If desired, the composition can be sweetened by adding a sweetener such as sorbitol, maltitol, hydrogenated glucose syrup and hydrogenated starch hydrolysate, high fructose corn syrup, cane sugar, beet sugar, pectin, or sucralose.

Nutraceutical compositions disclosed herein can be in the form of a solution. For example, the nutraceutical formulation can be provided in a medium, such as a buffer, a solvent, a diluent, an inert carrier, an oil, or a cream. In some examples, the formulation is present in an aqueous solution that optionally contains a non-aqueous co-solvent, such as an alcohol. The nutraceutical composition can also be in the form of powder, paste, jelly, capsule, or tablet. Lactose and corn starch are commonly used as diluents for capsules and as carriers for tablets. Lubricating agents, such as magnesium stearate, are typically added to form tablets.

Any food compositions may be formulated for a suitable administration route, for example, oral administration. For oral administration, the composition can take the form of, for example, tablets or capsules, prepared by conventional means with acceptable excipients such as binding agents (for example, pre-gelatinized maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Also included are bars and other chewable formulations.

In some aspects, the food compositions can be in a liquid form and the one or more edible carriers can be a solvent or dispersion medium comprising but not limited to, ethanol, polyol (e.g., glycerol, propylene glycol, liquid polyethylene glycol), lipids (e.g., triglycerides, vegetable oils, liposomes) or combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof. In many cases, it will be advisable to include an isotonic agent, such as, for example, sugars, sodium chloride or combinations thereof.

Liquid preparations for oral administration can take the form of, for example, solutions, syrups or suspensions, or they can be presented as a dry product for constitution with water or other suitable vehicle before use. In one aspect, the liquid preparations can be formulated for administration with fruit juice. Such liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates, benzoate or sorbate).

The health food compositions described herein may further comprise one or more second therapeutic agents, including those described herein.

The present disclosure also provides compositions of medical food products, use in improving basic condition during or in the risk of virus infection. A medical food product is a food product formulated to be consumed or administered enterally. Such a food product is usually used under the supervision of a physician for the specific dietary management of a target disease, such as those described herein. In some instances, such a medical food composition is specially formulated and processed (as opposed to a naturally occurring foodstuff used in a natural state) for a patient in need of the treatment (e.g., human patients who suffer from illness or who requires use of the product as a major active agent for alleviating a disease or condition via specific dietary management.) In some examples, a medical food composition described herein is not one of those that would be simply recommended by a physician as part of an overall diet to manage the symptoms or reduce the risk of a disease or condition.

Any of the medical food compositions described herein, comprising the diphenyl compound and at least one carrier (e.g., those described herein), can be in the form of a liquid solution; powder, bar, wafer, a suspension in an appropriate liquid or in a suitable emulsion, as detailed below. The at least one carrier, which can be either naturally-occurring or synthetic (non-naturally occurring), would confer one or more benefits to the composition, for example, stability, bioavailability, and/or bioactivity. Any of the carriers described herein may be used for making the medical food composition. In some aspects, the medical food composition may further comprise one or more additional ingredients selected from the group including, but not limited to natural flavors, artificial flavors, major trace and ultra-trace minerals, minerals, vitamins, oats, nuts, spices, milk, egg, salt, flour, lecithin, xanthan gum and/or sweetening agents. The medical food composition may be placed in a suitable container, which may further comprise at least an additional therapeutic agent such as those described herein.

Any of the compositions disclosed herein, comprising or consisting essentially of the one or more diphenyl compounds as also disclosed herein, may further comprise vitamin C and pharmaceutically acceptable carriers, for example, sodium carboxymethyl starch; cellulose derivatives such as methyl and ethyl cellulose; gelatin; inorganic compounds such as light magnesia, talc and the like; oils such as vegetable oils, sesame oil and the like; package agent such as β-cyclodextrin; and glycols such as polyethylene glycol. Such a composition may be formulated for oral administration, for example, in tablet format. To make tablets, the composition can be made into an infusion tablet together with a disintegrant composed of a mixture of sodium bicarbonate and tartaric acid or citric acid. When it comes into contact with water, carbon dioxide gas is generated to disintegrate the tablet rapidly

Alternatively, the compositions may comprise or consisting essentially of the one or more diphenyl compounds and one or more suitable pharmaceutically acceptable excipients to formulate it to an injectable composition. Examples of the pharmaceutically acceptable excipients include isotonic saline, 5% dextrose in water, water for injection, camellia oil, soybean oil, sesame oil, ethanol, glycerol, propylene glycol, polyethylene glycol, benzyl benzoate; TWEEN 80, lecithin, sodium deoxycholate, egg yolk, phospholipids, polyvinylpyrrolidone; acetate, sodium acetate, and sodium citrate, lactic acid, tartaric acid and sodium, disodium hydrogen phosphate, sodium dihydrogen phosphate, sodium bicarbonate and sodium carbonate; gelatin, sodium carboxymethylcellulose, pectin, sorbitol solution; creatinine, glycine; sodium sulfite, sodium bisulfite, sodium metabisulfite, sodium thiosulfate, benzyl alcohol, butyl paraben, ethyl phenol and chlorobutanol; lidocaine; cephalin, soya lecithin, typically in the form of injectable preparation for the powder, suspension type injectable compositions.

In other examples, any of the compositions disclosed herein may be formulated as aerosol formulations, for example, using a polyvinyl chloride paste resin, dibutyl phthalate, dioctyl phthalate, calcium stearate, zinc stearate formulated aerosols.

### Coronaviruses and Coronavirus Diseases

Coronaviruses are large, roughly spherical particles with unique surface projections. Their size is highly variable and generally is an average diameter of 120 nm. Extreme sizes are known from 50 to 200 nm in diameter. The total molecular weight is on average 40,000 kDa. They are enclosed in an envelope embedded with a number of protein molecules. The lipid bilayer envelope, membrane proteins, and nucleocapsid protect the virus when it is outside the host cell. Coronaviruses are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives. The scientific name Coronavirus was accepted as a genus name by the International Committee for the Nomenclature of Viruses in 1971. Coronaviruses form the subfamily Orthocoronavirinae, which is one of two sub-families in the family Coronaviridae, order Nidovirales, and realm Riboviria. They are divided into the four genera: Alphacoronavirus, Betacoronavirus, Gammacoronavirus and Deltacoronavirus. Alphacoronaviruses and betacoronaviruses infect mammals, while gammacoronaviruses and deltacoronaviruses primarily infect birds. As of 2020, 45 species are officially recognized. Six species of human coronaviruses are known, with one species subdivided into two different strains, making seven strains of human coronaviruses altogether.

Coronaviruses vary significantly in risk factor. Some can kill more than 30% of those infected, such as MERS-COV, and some are relatively harmless, such as the common cold. Coronaviruses can cause colds with major symptoms, such as fever, and a sore throat from swollen adenoids. Coronaviruses can cause pneumonia (either direct viral pneumonia or secondary bacterial pneumonia) and bronchitis (either direct viral bronchitis or secondary bacterial bronchitis). The human coronavirus discovered in 2003, SARS-COV, which causes severe acute respiratory syndrome (SARS), has a unique pathogenesis because it causes both upper and lower respiratory tract infections. Another potentially severe form of infection is caused by SARS-COV2, resulting in COVID-19, whose symptoms include fever, cough, fatigue, shortness of breath or breathing difficulties, and loss of smell and taste. While most people contracting COVID-19 have mild symptoms, some do develop acute respiratory distress syndrome (ARDS) possibly precipitated by cytokine storm, multi-organ failure, septic shock, and blood clots. For COVID-19 patients, longer-term damage to organs (in particular lungs and heart) has been observed, and there is concern about a significant number of patients who have recovered from the acute phase of the disease but continue to experience a range of effects including severe fatigue, memory loss and other cognitive issues, low grade fever, muscle weakness, breathlessness and other symptoms for months afterwards. The incubation period for COVID-19 may range from one to fourteen days. SARS-COV2 spreads very easily and sustainably through the air, primarily via small droplets and sometimes in aerosols, as an infected person breathes, coughs, sneezes, talks, or sings. It may also be transmitted via contaminated surfaces, although this has not been conclusively demonstrated. It can spread from an infected person for up to two days prior to symptom onset and from people who are asymptomatic. People remain infectious for seven to twelve days in moderate cases and up to two weeks in severe cases. The standard method of diagnosis is by real-time reverse transcription polymerase chain reaction (rRT-PCR) from a nasopharyngeal swab. Chest CT imaging may also be helpful for diagnosis in individuals where there is a high suspicion of infection based on symptoms and risk factors, however guidelines do not recommend using it for routine screening.

Intense research has been directed at developing vaccines and drug therapies to treat, prevent or reduce the severity of human coronavirus infections. Drug therapies are seeking to block different steps involving viral replication, proliferation and/or host cell entry. Drug targets can roughly be split into virus-based or host-based. Virus based drug targets include, but not limited to, various viral structural and non-structural proteases and polymerases, such as spike(S) glycoprotein, envelope (E) protein, membrane (M) protein, the nucleocapsid (N) protein, and RNA-Dependent RNA Polymerase. Host based targets include, but not limited to, virus entry proteins, host immune response and other control steps in host cellular machineries, such as Angiotensin I Converting Enzyme 2 (ACE2), Transmembrane Serine Protease 2 (TMPRSS2), Cathepsin L, Phosphatidylinositol 3-Phosphate 5-Kinase (PIKfyve), Adaptor-Associated Kinase 1 (AAK1) and Cyclin G-Associated Kinase (GAK). See Gil et al, “COVID-19: Drug Targets and Potential Treatments”, J. of Med. Chem. ePub, 0c006060, the entire content of which is incorporated by reference herein.

### Definitions

The present disclosure provides uses of any of the diphenyl compounds disclosed herein or any of the compositions comprising such for inhibiting, treating, reducing, prohibiting and preventing the viral load, the viral replication, the viral proliferation, and/or reducing morbidity or mortality in the clinical outcomes, in patients suffering from a coronavirus infection, for example, SARS, MERS, and coronavirus disease COVID-19. In yet another aspect, the present disclosure further provides methods of reducing the risk that an individual will develop a pathological coronavirus infection that has clinical sequelae. The methods generally involve administering a therapeutically effective amount of a therapeutically effective amount of the composition herein.

COVID-19 is a disease caused by severe acute respiratory syndrome coronavirus 2 (SARS-COV-2), previously known as 2019 novel coronavirus. In other instances, the patient may have an infection caused by another coronavirus such as severe acute respiratory syndrome coronavirus (SARS-COV) or middle east respiratory syndrome coronavirus (MERS-COV).

The method disclosed herein comprises administering to a subject in need thereof an effective amount of one or more diphenyl compounds or any of the compositions comprising such as described herein.

The term “about” or “approximately” used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein, the term “treat” refers to the application or administration of a composition including one or more active agents to a subject, who is in need of the treatment, for example, having a target disease or disorder, a symptom of the disease/disorder, or a predisposition toward the disease/disorder, with the purpose to cure, heal, alleviate, relieve, alter, remedy, ameliorate, improve, prevent or affect the disorder, the symptom of the disease, or the predisposition toward the disease or disorder.

Alleviating a target disease/disorder includes delaying the development or progression of the disease, or reducing disease severity. Ameliorating a target disease/disorder includes improving the condition, halting or reversing the progression of the disease. Alleviating or ameliorating a disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

To achieve any of the intended therapeutic effects described herein, an effective amount of a composition herein may be administered to a subject in need of the treatment via a suitable route.

As used herein, “an effective amount” refers to the amount of each active agent required to confer therapeutic effect on the subject, either alone or in combination with one or more other active agents, such as one or more of the second therapeutic agents described herein. In some aspects, the therapeutic effect is improvement of basic conditions of virus infection. In some aspects, the therapeutic effect is alleviating one or more symptoms associated with any of the infection by the virus described herein.

Determination of whether an amount of the composition as described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration, genetic factors and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration and/or route of administration may be determined and adjusted over the course of therapy, and is generally, but not necessarily, based on treatment and/or suppression and/or amelioration and/or delay of a target disease/disorder. Alternatively, sustained continuous release formulations of a composition as described herein may be appropriate. Various formulations and devices for achieving sustained release are known in the art.

In some aspects, the effective amount can be a prophylactically effective amount (e.g., amount effective for inhibiting, treating, reducing the viral load, and/or reducing morbidity or mortality in a subject suffering from the viral infection in need of such an effect) to reduce the risk of having coronavirus infection.

The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, cat, cow, chicken, etc. The subject may have or be suspected of having infection by a coronavirus. In some examples, the subject may have confirmed of having a coronavirus infection or COVID-19, or suspected of having a coronavirus infection by displaying symptoms or discomforts, such as elevated temperature, fever, cough, dyspnea, chills, persistent tremor, muscle pain, headache, sore throat, and loss of taste or smell (see ageusia and anosmia), or other symptoms of a viral pneumonia.

A human subject who needs the treatment may be a human patient having, at risk for, or suspected of having a target disease/disorder, such as an infection by virus, or by Coronavirus. A human subject can be of any age, such as an infant, a toddler, a pre-schooler, a pre-teen, a teenager, a youth, or an adult. A human subject may have an age from 0 to 2 years old, from 2 to 12 years old, from 12 to 16 years old, from 16 to 18 years old, from 18 to 21 years old, from 21 to 30 years old, from 30 to 50 years old, from 50 to 60 years old, or older than 60 years. A human subject can be male, female, or transgender.

In some instances, the diphenyl compounds or the composition comprising such may be administered to the subject over several days to several weeks or longer at a suitable interval, depending on the condition, the treatment is sustained until a desired suppression of symptoms occurs or until sufficient therapeutic levels are achieved to alleviate a target disease or disorder, or a symptom thereof. An exemplary dosing regimen comprises administering one or more initial doses at a suitable interval over a suitable period. If necessary, multiple maintenance doses can be given to the subject at a suitable interval over a suitable period of time. However, other dosage regimens may be useful, depending on the pattern of pharmacokinetic decay that the practitioner wishes to achieve. For example, dosing from one to twenty-four times a day or a week is contemplated. In some aspects, the dose may be administered to the patient 1, 2, 3, 4, or 5 times per day. In particular, the composition may be administered orally.

In some aspects, dosing frequency can be continuously for the period medically or therapeutically needed, every one hour, every two hours, four times a day, three times a day, twice a day, once a day, once every other day, once every week, once every 2 weeks, once every 4 weeks, once every 2 months, once every 3 months or only given once. The dosing regimen can vary over time.

The particular dosage regimen, i.e., dose, timing and repetition, will depend on the particular individual and that individual's medical history, as well as the properties of the individual agents (such as the half-life of the agent, and other considerations well known in the art).

Conventional methods, known to those of ordinary skill in the art of medicine, can be used to administer the composition (e.g., a pharmaceutical composition, a health food composition, a nutraceutical composition or a medical food composition) to the subject, depending upon the type of viral infection disease to be treated or the site of the disease. This composition can also be administered via other conventional routes, e.g., administered orally, parenterally, by inhalation spray, topically, rectally, nasally, buccally, or via an implanted reservoir. The term “parenteral” as used herein includes subcutaneous, intracutaneous, intravenous, intramuscular, intraarticular, intra-arterial, intrasynovial, intrasternal, intrathecal, intralesional, and intracranial injection or infusion techniques.

The composition can be administered by pulmonary delivery system, that is, the active pharmaceutical ingredient is administered into lung. The pulmonary delivery system can be an inhaler system. In some aspects, the inhaler system is a pressurized metered dose inhaler, a dry powder inhaler, or a nebulizer. In some aspects, the inhaler system is with a spacer.

In some aspects, the pressurized metered dose inhaler includes a propellant, a co-solvent, and/or a surfactant. In some aspects, the propellant is selected from the group comprising of fluorinated hydrocarbons such as trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane. In some aspects, the co-solvent is selected from the group comprising of purified water, ethanol, propylene glycol, glycerin, PEG400, PEG 600, PEG 800 and PEG 1000. In some aspects, the surfactant or lubricants is selected from the group comprising of sorbitan trioleate, soya lecithin, lecithin, oleic acid, Polysorbate 80, magnesium stearate and sodium lauryl sulfate. In some aspects, the preservatives or antioxidants is selected from the group comprising of methylparaben, propylparaben, chlorobutanol, benzalkonium chloride, cetylpyridinium chloride, thymol, ascorbic acid, sodium bisulfite, sodium metabisulfite, sodium bisulfate, EDTA. In some aspects, the pH adjustments or tonicity adjustments is selected from the group comprising of sodium oxide, tromethamine, ammonia, HCl, H₂SO₄, HNO₃, citric acid, CaCl₂), CaCO₃.

In some aspects, the dry powder inhaler includes a disperse agent. In some aspects, the disperse agent or carrier particle is selected from the group comprising of lactose, lactose monohydrate, lactose anhydrate, mannitol, dextrose which their particle size is about 1-100 μm.

In some aspects, the nebulizer may include a co-solvent, a surfactant, lubricant, preservative and/or antioxidant. In some aspects, the co-solvent is selected from the group comprising of purified water, ethanol, propylene glycol, glycerin, PEG (e.g., PEG400, PEG600, PEG800 and/or PEG 1000).

In some aspects, the surfactant or lubricant is selected from the group comprising of sorbitan trioleate, soya lecithin, lecithin, oleic acid, magnesium stearate and sodium lauryl sulfate.

In some aspects, the preservative or antioxidant is selected from the group comprising of methylparaben, propylparaben, chlorobutanol, benzalkonium chloride, cetylpyridinium chloride, thymol, ascorbic acid, sodium bisulfite, sodium metabisulfite, sodium bisulfate, EDTA.

In some aspects, the nebulizer further includes a pH adjustment or a tonicity adjustment, which is selected from the group comprising of sodium oxide, tromethamine, ammonia, HCl, H₂SO₄, HNO₃, citric acid, CaCl₂), CaCO₃.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water-soluble antibodies can be administered by the drip method, whereby a pharmaceutical formulation the composition described herein and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the composition herein, can be dissolved and administered in a pharmaceutical excipient such as Water-for-Injection, 0.9% saline, or 5% glucose solution.

In one aspect, the composition is administered via a site-specific or targeted local delivery technique. Examples of site-specific or targeted local delivery techniques include various implantable depot sources of the compositions or local delivery catheters, such as infusion catheters, an indwelling catheter, or a needle catheter, endotracheal tube, endobronchial catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Treatment efficacy for a target disease/disorder can be assessed by methods well-known in the art.

### Combined Therapy

Also provided herein are combined therapies using any of the compositions described herein and a second therapeutic agent, such as those described herein. The term combination therapy, as used herein, embraces administration of these agents (e.g., the diphenyl compound and an antiviral agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route including, but not limited to, oral routes, intravenous routes, intramuscular, subcutaneous routes, direct absorption through mucous membrane tissues, and pulmonary delivery routes. The agents can be administered by the same route or by different routes. For example, a first agent (e.g., a composition described herein) can be administered by pulmonary delivery routes, and a second agent (e.g., an antiviral agent) can be administered intravenously.

Examples of the additional pharmaceutical agent selected from a viral entry inhibitor, a viral uncoating inhibitor, a viral reverse transcriptase inhibitor, a viral protein synthesis inhibitor, a viral protease inhibitor, a viral polymerase inhibitor, a viral integrase inhibitor, an interferon, or the combination thereof. Examples of viral entry inhibitor include, but are not limited to, maraviroc, enfuvirtide, ibalizumab, fostemsavir, plerixafor, epigallocatechin gallate, vicriviroc, aplaviroc, maraviroc, tromantadine, nitazoxanide, umifenovir, and podofilox. Examples of viral uncoating inhibitor include, but are not limited to, amantadine, rimantadine, and pleconaril. Examples of reverse transcriptase inhibitor include, but are not limited to zidovudine, didanosine, zalcitabine, stavudine, lamivudine, abacavir, emtricitabine, entecavir, truvada, nevirapine, raltegravir, and tenofovir disoproxil. Examples of viral protease inhibitor, include but are not limited to, fosamprenavir, ritonavir, atazanavir, nelfinavir, indinavir, saquinavir, saquinavir, famciclovir, fomivirsen, lopinavir, ribavirin, darunavir, oseltamivir, and tipranavir. Examples of viral polymerase inhibitor, include but are not limited to, amatoxins, rifamycin, cytarabine, fidaxomicin, tagetitoxin, foscarnet sodium, idoxuridine, penciclovir, sofosbuvir, trifluridine, valacyclovir, valganciclovir, vidarabine, and remdesivir. Examples of viral integrase inhibitor, include but are not limited to, raltegarvir, elvitegravir, dolutegravir, bictegravir, and cabotegravir. Examples of interferon, include but are not limited to, type I interferon, type II interferon, type III interferon, and peginterferon alfa-2a.

As used herein, the term “sequential” means, unless otherwise specified, characterized by a regular sequence or order, e.g., if a dosage regimen includes the administration of a composition and an antiviral agent, a sequential dosage regimen could include administration of the composition before, simultaneously, substantially simultaneously, or after administration of the antiviral agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the agents described herein.

Combination therapy can also embrace the administration of the agents described herein (e.g., the composition and an antiviral agent) in further combination with other biologically active ingredients (e.g., a different antiviral agent) and non-drug therapies.

It should be appreciated that any combination of a composition described herein and a second therapeutic agent (e.g., an antiviral agent) may be used in any sequence for treating a target disease. The combinations described herein may be selected on the basis of a number of factors, which include but are not limited to the effectiveness of inhibiting virus or at least one symptom associated with the virus infection.

### Kits for Treating Coronavirus Infection

The present disclosure also provides kits for use in treating coronavirus infection. Such kits can include one or more containers comprising the composition as described herein and optionally one or more of the second therapeutic agents as also described herein.

In some aspects, the kit can comprise instructions for use in accordance with any of the methods described herein. The included instructions can comprise, for example, a description of administration of the diphenyl compound and optionally a description of administration of the second therapeutic agent(s) to improve medical conditions of virus infection or in the risk of virus infection. The kit may further comprise a description of selecting an individual suitable for treatment based on identifying whether that individual has the disease or is at risk for the disease. In still other aspects, the instructions comprise a description of administering one or more agents of the disclosure to an individual at risk of virus infection.

The instructions relating to the use of the diphenyl compound to achieve the intended therapeutic effects generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk, or QR code) are also acceptable.

The label or package insert may indicate that the composition is used for the intended therapeutic utilities. Instructions may be provided for practicing any of the methods described herein.

The kits of this invention are in suitable packaging. Suitable packaging includes, but is not limited to, chambers, vials, bottles, jars, flexible packaging (e.g., sealed Mylar or plastic bags), and the like. Also contemplated are packages for use in combination with a specific device, such as an inhaler, nebulizer, ventilator, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some aspects, the invention provides articles of manufacture comprising contents of the kits described above.

### General Techniques

The practice of the present invention will employ, unless otherwise indicated, conventional techniques of virology, molecular biology (including recombinant techniques), microbiology, cell biology, biochemistry and immunology, which are within the skill of the art. Such techniques are explained fully in the literature. Without further elaboration, it is believed that one skilled in the art can, based on the above description, utilize the present invention to its fullest extent. The specific aspects provided herein are, therefore, to be construed as merely illustrative, and not limitative of the remainder of the disclosure in any way whatsoever. All publications cited herein are incorporated by reference for the purposes or subject matter referenced herein. Various aspects of the disclosure are discussed in detail below. While specific implementations are discussed, it should be understood that this is done for illustration purposes only. A person skilled in the relevant art will recognize that other components and configurations may be used without parting from the spirit and scope of the disclosure. It should be understood at the outset that although illustrative implementations of one or more aspects are illustrated below, the disclosed method may be implemented using any number of techniques. The disclosure should in no way be limited to the illustrative implementations, drawings, and techniques illustrated herein, but may be modified within the scope of the appended claims along with their full scope of equivalents.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense. Specifically, a series experiments as shown below are designed to determine if the diphenyl compound, such as ANAVEX1066 (AV1066), has prophylactic or therapeutic activity against SARS-COV-2. It would be appreciated by an artisan in in the art that these experiments are only for illustrative purpose, other methods or experimental designs routinely used in the antiviral studies are also applicable here and within the scope of the current disclosure.

### Example 1. Synthesis of Diphenyl Compounds

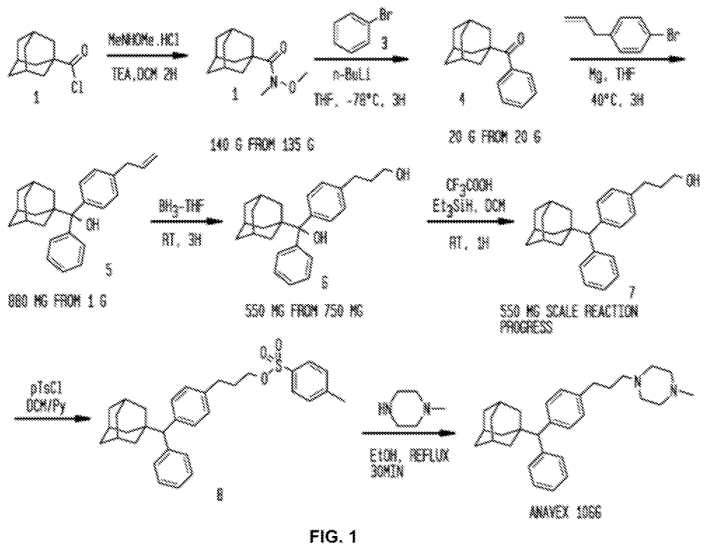

Various diphenyl compounds were synthesized in house as disclosed in Journal of Medicinal Chemistry (2012), 55 (22), 10241-10261. Its entire disclosure is incorporated herein by its full content. FIG. 1 provides a schematic scheme of the synthesis of one of the diphenyl compound, AV1066. The thus obtained crude diphenyl compound were dried under reduced pressure. Recrystallization from water yielded the pure diphenyl compound with purity over 95%.

### Example 2. Prophylactic Activity of the Diphenyl Compound against SARS-COV-2

The diphenyl compound used in this study was the dichloride salt of ANAVEX1066 (AV1066), an intermediate of ANAVEX 2-73 synthesis. Its solubility in water is 4 mg/ml, and in DMSO is 21 mg/ml. Materials and samples included:
Vero E6
T-175 tissue culture flask.
0.25% Trypsin-EDTA (1X).
Minimum Essential Medium (MEM) with Earle's salts and L-glutamine, VWR Cat #10-010-CV.
Dulbecco's Phosphate Buffered Saline 1X.
Fetal bovine serum (FBS), BioWest Cat #S1620, Heat inactivated, (Lot No: 055G17).
Greiner Bio-one TC treated PS sterile cell culture 12 well plates (Cat #07-000-691)
Virus: SARS-Related Coronavirus 2 Isolate USA-WA1/2020.
A1066: test concentrations of 8 μM, 4 μM, 2 μM, and 1 μM in triplicate (12 samples).
Control: equivalent volume of water or DMSO in triplicate (3 samples)
Remdesivir: 10 μM (3 samples), its solubility in DMSO is 100 mg/mL

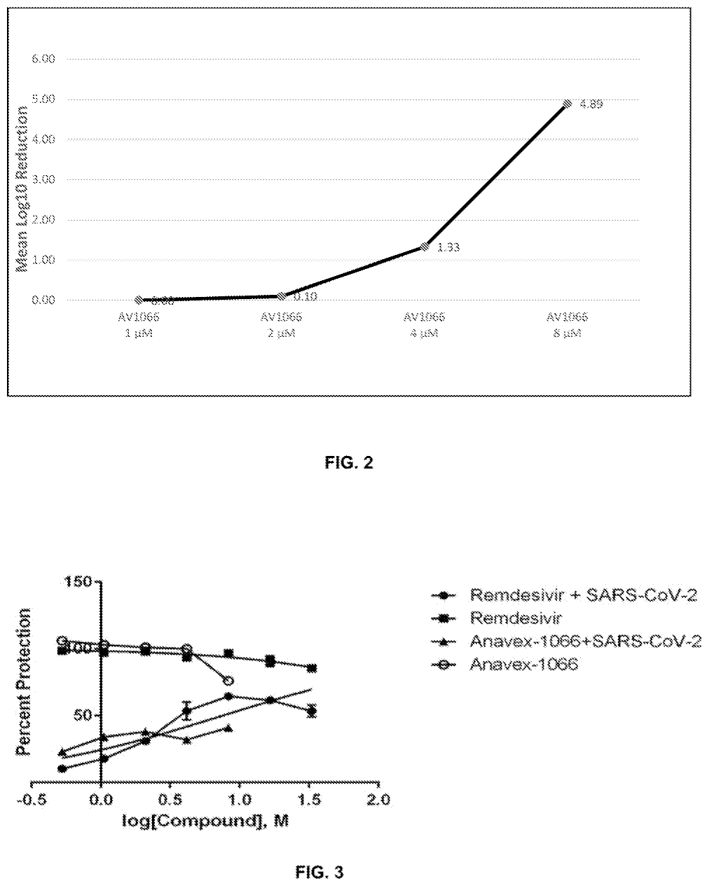

The prophylactic methodology included testing steps of:
1) Seed 2.5×105 Vero E6 cells to each well in 12-well plate.
2) Allow cells to grow overnight. No swirling, otherwise cell density will be lower in the center of the well. Cells should be 70-90% confluent the day of infection.
3) Next day remove media, wash each well once with 1 ml PBS
4) Add:
   a) 0.5 ml fresh media (MEM+2% FBS+1% P−S)+0.5 ml AV1066 (final concentrations in 1 ml total: 8 μM, 4 μM, 2 μM, and 1 μM
   b) 0.5 ml fresh media+0.5 ml Control (Sample equivalent volume amount in μl)
   c) 0.5 ml fresh media (MEM+2% FBS+1% P−S)+0.5 ml Remdesivir (final concentrations in 1 ml total: μM
5) Incubate for 2 hr at 37° C. and then remove well components
6) Add SARS-COV-2, 2.5×104/0.2 ml (MOI=0.1) diluted in MEM+2% FBS, to each well.
7) Incubate for 1 hr at 37° C. Swirl the plates every 15 min
8) After 1 hr, remove virus, add:
   a.) 0.5 ml fresh media (MEM+2% FBS+1% P−S)+0.5 ml AV1066 (final concentrations in 1 ml total: 8 μM, 4 μM, 2 M, and 1 μM
   b.) 0.5 ml fresh media+0.5 ml Control (Sample equivalent volume amount in μl)
   c.) 0.5 ml fresh media (MEM+2% FBS+1% P−S)+0.5 ml Remdesivir (final concentrations in 1 ml total: μM
9) Incubate for 2 days at 37° C.
10) After 2 days, harvest components from each well and perform TCID50 assay. Residual virus was measured by TCID50 assay in 96-well plate format with 3 wells per dilution of virus. 10-fold serial dilutions (10⁻¹ to 10⁻⁷) of collected samples (treated, control, or virus only) were used to infect Vero E6 cells in a 96-well plate. The cell plates were incubated at 37° C., 5% CO₂, with humidity for an additional 3 days. After 2 days, an MTT assay was used to determine viability of cells. The virus endpoint titer was determined using the Reed-Muench formula and expressed as log 10 TCID50/ml. Viral log reduction is calculated by the below formula, and the results were summarized as Table 2 and FIG. 2.
Log Reduction=Log10 (C/T)
C=mean viable virus recovered from untreated control
T=mean viable virus recovered from compound treated samples

As shown in Table 2 and FIG. 2, AV1066 exhibited antiviral activity against SARS-COV-2 in a dose dependent manner. At the maximum concentration (8 μM), a 4.89 log reduction was observed when compared to the untreated control. As a comparison, 10 M remdesivir demonstrated a 5.72 log indicating an inhibition efficacy similar to remdesivir.

TABLE 2
SARS-CoV-2 Titer Reduction by AV1066
Antiviral COVID-19 Activity with AV1066
Treatment 2 Hours Pre-Infection

| Sample | Mean Log₁₀ Reduction |
|---|---|
| Virus | — |
| Control | |
| Positive control: | 5.72 |
| Remdesivir | |
| 10 μM | |
| AV1066 | 0.00 |
| 1 μM | |
| AV1066 | 0.10 |
| 2 μM | |
| AV1066 | 1.33 |
| 4 μM | |
| AV1066 | 4.89 |
| 8 μM | |

### Example 3. Therapeutic Activity of the Diphenyl Compound against SARS-COV-2 Infection

The diphenyl compound, AV1066 was further tested for its therapeutic activity against SARS-COV-2 infection. In this experiment, the same set of materials and concentrations were used as in Example 2, and the testing steps include:
1) Seed 2.5×10⁵ Vero E6 cells to each well in 12-well plate.
2) Allow cells to grow overnight. No swirling, otherwise cell density will be lower in the center of the well. Cells should be 70-90% confluent the day of infection.
3) Next day remove media, wash each well once with 1 ml PBS
4) Add SARS-COV-2, 2.5×10⁴/0.2 ml (MOI=0.1) diluted in MEM+2% FBS, to each well.
5) Swirl the plates every 15 min
6) After 1 h remove virus, add 0.5 ml of fresh media media (MEM+2% FBS+1% P−S).
7) Incubate for 1 hr at 37° C.
8) After 1 hr, add:
   a) 0.5 ml AV1066 (final concentrations in 1 ml total: 8 μM, 4 μM, 2 μM, and 1 μM
   b) 0.5 ml Control (Sample equivalent volume amount in μl)
   c) 0.5 ml Remdesivir (final concentrations in 1 ml total: μM
9) Incubate for 2 days at 37° C.
10) After 2 days, harvest components from each well and perform TCID50 assay. TCID50 assay was performed in 96-well plate format with 3 wells per dilution of virus. 10-fold serial dilutions (10⁻¹ to 10⁻⁷) of collected samples (treated, control, or virus only) were used to infect Vero E6 cells in a 96-well plate. The cell plates were incubated at 37° C., 5% CO₂, with humidity for an additional 3 days. After 2 days, an MTT assay was used to determine viability of cells. The virus endpoint titer was determined using the Reed-Muench formula and expressed as log 10 TCID50/ml.

The invention claimed is:

1. A method for inhibiting or preventing a coronavirus infection, comprising administering to a subject in need thereof an effective amount of a diphenyl compound of Formula (I), an intermediate thereof, a metabolite thereof, an isomer thereof, an ester thereof, a pharmaceutically acceptable salt thereof, and a combination thereof. 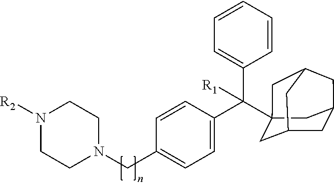
wherein
n is an integer of 1, 2 or 3;
R₁ and R₂ are each independently selected from the group consisting of hydrogen, C1-6 alkyl or C2-6 alkenyl, and C2-6 alkynyl.

2. The method of claim 1, wherein the effective amount of the diphenyl compound is sufficient to inhibit replication or proliferation of a coronavirus.

3. The method of claim 1, wherein the subject is having or suspected of having a coronavirus infection caused by a severe acute respiratory syndrome coronavirus 2 (SARS-COV-2), a severe acute respiratory syndrome coronavirus (SARS-CoV), a Middle East respiratory syndrome coronavirus (MERS-COV), or a combination thereof.

4. The method of claim 1, wherein the diphenyl compound is ANAVEX 1066, its metabolites, its isomers, its pharmaceutically acceptable salts, or any combinations thereof.

5. A method for treating a coronavirus disease in a subject, comprising administering to the subject in need thereof an effective amount of a diphenyl compound of Formula (I), an intermediate thereof, a metabolite thereof, an isomer thereof, an ester thereof, a pharmaceutically acceptable salt thereof, and a combination thereof. 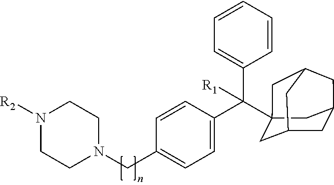
wherein
n is an integer of 1, 2 or 3;
R₁ and R₂ are each independently selected from the group consisting of hydrogen, C1-6 alkyl or C2-6 alkenyl, and C2-6 alkynyl.

6. The method of claim 5, wherein the subject is having or suspected of having a coronavirus disease selected from severe acute respiratory syndrome (SARS), coronavirus disease 2019 (COVID-19), Middle East respiratory syndrome (MERS), or a combination thereof.

7. The method of claim 5, wherein the diphenyl compound is ANAVEX 1066, its metabolites, its isomers, its pharmaceutically acceptable salts, or any combinations thereof.

8-19. (canceled)

20. A method for alleviating or ameliorating symptoms related to a coronavirus disease in a subject, comprising administering to the subject in need thereof an effective amount of a diphenyl compound of Formula (I), an intermediate, a metabolite, an enantiomer, an isomer, an ester, or a pharmaceutically acceptable salt, or any combination thereof. 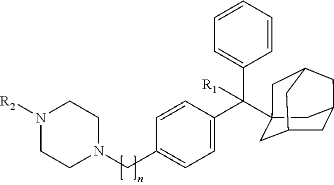
wherein
n is an integer of 1, 2 or 3;
R1 and R2 are each independently selected from the group consisting of hydrogen, C1-6 alkyl or C2-6 alkenyl, and C2-6 alkynyl.

21. The method of claim 20, wherein the symptoms related to a coronavirus disease comprises fever, chills, cough, shortness of breath, difficulty breathing, fatigue, muscle ache, body ache, headache, loss of taste or smell, sore throat, congestion or runny nose, nausea, vomiting, diarrhea, trouble breathing, persistent pain or pressure in the chest, new confusion, inability to wake or stay awake, or bluish lips or face.

22. The method of claim 21, wherein the coronavirus disease is severe acute respiratory syndrome (SARS), coronavirus disease 2019 (COVID-19), Middle East respiratory syndrome (MERS), or a combination thereof.

23. The method of claim 20, wherein the diphenyl compound is AV1066, its metabolites, its isomers, its pharmaceutically acceptable salts, or any combinations thereof.

24. The method of claim 20, wherein the diphenyl compound is administered orally or parenterally.

25. The method of claim 20, wherein the diphenyl compound is administered in a composition in the form of a pharmaceutical composition, a health food composition, or a medical food composition.

26. (canceled)

27. The method of claim 20, wherein the subject is a human being.

28. (canceled)

29. The method of claim 20, wherein the effective amount of the diphenyl compound administered to the subject is from about 0.5 mg to about 1000 mg.

30. (canceled)

31. The method of claim 20, wherein the diphenyl compound is administered to the subject at a frequency of three times per day to once per week.

32. The method of claim 20, wherein the subject has undergone or is undergoing treatment with one or more additional anti-viral agents.

33. The method of claim 1, wherein the diphenyl compound is administered in a composition in the form of a pharmaceutical composition, a health food composition, or a medical food composition.

34. The method of claim 5, wherein the diphenyl compound is administered in a composition in the form of a pharmaceutical composition, a health food composition, or a medical food composition.

35. The method of claim 1, wherein the effective amount of the diphenyl compound administered to the subject is from about 0.5 mg to about 1000 mg.
